(19) United States
(12) Patent Application Publication
(10) Pub. No.: US 2025/0312375 A1
(43) Pub. Date: Oct. 9, 2025

(54) EXTRACELLULAR VESICLE DEPLETED BLOOD FRACTIONS

(71) Applicant: GRIFOLS WORLDWIDE OPERATIONS LIMITED, DUBLIN (IE)

(72) Inventors: Ana Maria ORTIZ FERNANDEZ, BARCELONA (ES); Carla MINGUET, BARCELONA (ES); Montserrat COSTA RIEROLA, BARCELONA (ES); Eulalia MARTÍ PUIG, BARCELONA (ES); Ana GÁMEZ VALERO, BARCELONA (ES); Maria SOLAGUREN-BEASCOA NEGRE, BARCELONA (ES)

(21) Appl. No.: 18/854,527

(22) PCT Filed: Apr. 5, 2023
(86) PCT No.: PCT/EP2023/058937

§ 371 Date: Oct. 5, 2024

(30) Foreign Application Priority Data
Apr. 6, 2022 (EP) 22382330.3

(51) Int. Cl. A61K 35/16; A61P 25/28

(52) U.S. Cl. CPC A61K 35/16; A61P 25/28

(57) ABSTRACT

Disclosed herein are human blood fractions depleted of disease-causing extracellular vesicles, prepared by plasma exchange, that may find use in the treatment of a condition selected from the group consisting of neurodegenerative disease, autoimmune disease, cardiovascular disease, renal disease, and liver disease. In particular, the depleted blood fractions may find use in treating neurodegenerative diseases such as Parkinson's Disease or Alzheimer's Disease.

### CROSS-REFERENCE TO RELATED APPLICATIONS

This application is a National Stage Application under 35 U.S.C. 371 of PCT Application No. PCT/EP2023/058937, filed on Apr. 5, 2023, which is an International Application that claims priority from a GB Patent Application No. 22382330.3, filed on Apr. 6, 2022, the contents of each of the above applications are incorporated herein by reference in their entireties.

### DESCRIPTION

### Field of the Invention

The present invention provides for methods of decreasing extracellular vesicle load in patient plasma, and methods of treating pathologies in which said extracellular vesicles are implicated.

### Background

Extracellular vesicles are lipid bilayer-delimited particles that are released from cells into the extracellular space by budding from the plasma membrane, or alternatively by invagination of the endosomal membrane and maturation into a multivesicular body that fuses with the plasma membrane so as to release its contents. Extracellular vesicles are thought to provide a means of intercellular communication and of transmission of macromolecules between cells.

Extracellular vesicles have been implicated as contributing factors in the development of several diseases owing to their role in the delivery of proteins, lipids, mRNA, miRNA and DNA from one cell to another. Packaging of extracellular vesicles appears to be indiscriminate, as such extracellular vesicles can provide for the delivery of both “good” and ‘bad’ cargo to their target cells. Extracellular vesicles have been reported to contain numerous disease-associated cargos, for example:
miRNAs in the case of cancer, and
neurodegenerative-associated peptides, such as Al3, tau, prions, and alpha-synuclein.

Whilst knowledge about extracellular vesicles is growing, the means by which disease-associated factors spread between cells remains poorly understood even though extracellular vesicles are implicated in such processes/transmission. Moreover, a thorough understanding of the mechanism whereby particular cargos [proteins, RNAs, etc.] are sorted into particular extracellular vesicles remains elusive. Consequently, traditional pharmaceutical targeting of extracellular vesicles continues to be challenging owing to structural non-homogeneity in the various different types of extracellular vesicles, and the aforementioned knowledge gaps around the molecular mechanisms by which extracellular vesicles are packaged and spread.

As such, there remains a need for therapies that can consistently and reproducibly reduce “bad” extracellular vesicle loads in patients thereby providing a reliable means to down-regulate the spread of extracellular vesicles and the consequent pathologies/diseases associated therewith.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Treatment of the Invention

In a first aspect, the present invention provides for extracellular vesicle depleted human blood fractions for use in the treatment of a condition selected from the group consisting of: neurodegenerative disease, autoimmune disease, cardiovascular disease, renal disease, liver disease, and combinations thereof, wherein blood obtained from a patient diagnosed with the condition is subjected to plasma exchange so as to deplete the extracellular vesicle content prior to being re-administered to said patient.

In a second aspect, the present invention provides for use of plasma exchange for removing extracellular vesicles from a patient in need thereof, wherein the patient is a patient diagnosed with a condition selected from the group consisting of cardiovascular disease, renal disease, liver disease, neurodegenerative disease, autoimmune disease, and combinations thereof, and further wherein no binding agent that specifically binds the extracellular vesicles is utilised in the plasma exchange process.

By “blood fraction” it is meant any derivative of whole blood that has been processed or fractionated to alter the natural concentrations of red blood cells, white blood cells, platelets, or plasma in whole blood. In some embodiments, “blood fraction” shall be construed to mean a derivative of whole blood that has been processed to reduce or alter the plasma component alone. Suitable methods of generating blood fractions from whole blood include, without limitation, plasmapheresis.

By “depleted” the present specification means depleted of disease-causing extracellular vesicles compared to a blood sample from the patient that had not been subjected to the plasma exchange procedure. In some embodiments, depletion may equate to a greater than 90% reduction in disease-causing extracellular vesicles compared to a blood sample from the patient that had not been subjected to the plasma exchange procedure. For example, depletion may equate to a reduction greater than 80%, 70%, 60%, 50%, 40%, 30%, 20%, or 10% in disease-causing extracellular vesicles compared to a blood sample from the patient that had not been subjected to the plasma exchange procedure. Disease causing extracellular vesicles may be identified by targeting particular sets of biomarkers outlined in the Detailed Examples of the Invention and assessing the contents of the extracellular vesicles. Such techniques are within the repertoire of a person of skill in the art.

In some embodiments, prior to being treated with the extracellular vesicle depleted human blood fraction of the present invention the patient diagnosed with the condition has been assessed to determine the presence or absence of disease-causing extracellular vesicles in their plasma. For example, in certain embodiments the patient may have tested positive for disease-causing extracellular vesicles prior to the treatment of the present invention.

In other embodiments, the patient diagnosed with the condition may be non-responsive/refractory to the standard of care/first line pharmacological treatment (for said condition) with small molecule drugs/biological drugs prior to treatment with the extracellular vesicle depleted human blood fraction of the present invention. The present invention envisages that the innovative treatments disclosed herein could be administered complimentarily to the standard of care treatments as opposed to the two being mutually exclusive.

As used herein, the term “plasma exchange” means a procedure in which a patient's blood is passed through a device, for example a plasmapheresis device, and the plasma component filtered by the device is removed and discarded. Red blood cells and other non-filtered blood fractions, optionally along with replacement fluid such as fresh frozen plasma or albumin, are reinfused back into the patient. Traditionally, the efficacy of plasma exchange is proportional to the plasma volume removed in relation to the patient's total plasma volume.

Advantageously, with respect to both aspects of the present invention, subjecting the patient's blood to plasma exchange without the incorporation of any specific extracellular vesicle binding agents (such as antibodies, antibody fragments, aptamers, etc.) circumvents problems associated with non-reproducible binding of structurally non-homogeneous extracellular vesicles. Moreover, obviating the need for expensive binding reagents greatly reduces the cost of any treatment for patients and health care providers. As used herein, “extracellular vesicle binding agent” shall be construed to mean materials capable of selectively binding with the extracellular vesicles over other plasma components and include antibodies, antibody fragments, other binding proteins, aptamers, and the like.

For the avoidance of any doubt, plasma exchange as referred to within this specification does not comprise the utilisation of any selective extracellular vesicle binding agents as part of the process.

By “extracellular vesicle”, the present specification means lipid bilayer-delimited particles that contain macromolecules such as proteins, lipids, nucleic acids, and combinations thereof that are released from cells into the intracellular space and have a diameter of 20-200 nm as determined by cryo-transmission electron microscopy (CRYO-TEM) analysis at −179° C. and an accelerating voltage of 200 kV. In one embodiment, the extracellular vesicles may be between 30-200 nm in diameter, 30-150 nm in diameter, 30-120 nm in diameter, 30-100 nm in diameter, 40-200 nm in diameter, 40-150 nm in diameter, 40-120 nm in diameter, or 40-100 nm in diameter. Extracellular vesicles as referred to in the present specification may be enriched in tetraspanins CD9, CD63, CD81, and combinations thereof.

The extracellular vesicles as referred to in the present specification may be 30-200 nm in diameter, 30-150 nm in diameter, 30-120 nm in diameter, or 30-100 nm in diameter as determined by cryo-transmission electron microscopy analysis [at −179° C. and an accelerating voltage of 200 kV] and may be enriched in tetraspanins CD9, CD63, CD81, and combinations thereof. For example, the extracellular vesicles as referred to in the present specification may be 40-200 nm in diameter, 40-150 nm in diameter, 40-120 nm in diameter, or 40-100 nm in diameter as determined by cryo-transmission electron microscopy analysis at −179° C. and may be enriched in tetraspanins CD9, CD63, CD81, and combinations thereof.

### Neurodegenerative Disease

In one embodiment, the depleted blood fraction of the present invention is for use in the treatment of neurodegenerative disease. Similarly, with respect to the second aspect of the present invention, the use of plasma exchange for removing extracellular vesicles in a patient may be utilised where the patient is a patient diagnosed with a neurodegenerative disease.

For example, the neurodegenerative disease may be selected from the group consisting of Parkinson's disease, synucleinopathies, Alzheimer's disease, Mild Cognitive Impairment, Diffuse Lewy body disease, Dementia with Lewy bodies type, amyotrophic lateral sclerosis, Pick's disease, tauopathies, trinucleotide repeat expansion diseases such as (without limitation) Huntington's disease and spinocerebellar ataxias, Creutzfeldt-Jakob disease, frontotemporal dementia, and combinations thereof.

In preferred embodiments, the neurodegenerative disease may be selected from the group consisting of Parkinson's disease, Alzheimer's disease, and Huntington's disease. For example, the neurodegenerative disease may be Alzheimer's disease or Parkinson's Disease.

In one embodiment, the neurodegenerative disease is Alzheimer's disease. The Alzheimer's disease may be mild to moderate. For example, the patient suffering from Alzheimer's disease may have a mini mental state examination (MMSE) score of between 10-26, such as for example 18-26. For example, the Alzheimer's disease patient may have a MMSE score of between 10-15, 15-26, or 18-26.

In some embodiments, the Alzheimer's disease patient may have an extracellular vesicle content different from a non-cognitively impaired control sample. For example, the Alzheimer's disease patient may have an extracellular vesicle content in which at least one of the proteins listed in Table 1 (of Example 2) is present in a higher or a lower concentration compared to a non-cognitively impaired control sample. In some embodiments, at least one of the proteins indicated as down regulated (in Table 1) is present in a lower concentration compared to a non-cognitively impaired control sample. In some embodiments, at least one of the proteins indicated as up regulated (in Table 1) is present in a higher concentration compared to a non-cognitively impaired control sample.

In other embodiments, the Alzheimer's disease patient may have an extracellular vesicle content in which at least one protein selected from the group consisting of CLU, HSPA5, HSP90B1, CALR, PLPT, and SOD2 is present in a higher or lower concentration compared to a non-cognitively impaired control sample. For example, the Alzheimer's disease patient may have an extracellular vesicle content in which at least one protein selected from the group consisting of CLU, HSPA5, HSP90B1, CALR, PLPT, and SOD2 is present in a higher concentration compared to a non-cognitively impaired control sample. In yet a further embodiment, the Alzheimer's disease patient may have an extracellular vesicle content in which at least one protein selected from the group consisting of CLU and SOD2 is present in a higher or lower concentration compared to a non-cognitively impaired control sample. For example, the Alzheimer's disease patient may have an extracellular vesicle content in which at least one protein selected from the group consisting of CLU and SOD2 is present in a higher concentration compared to a non-cognitively impaired control sample.

In other embodiments, the Alzheimer's disease patient may have an extracellular vesicle content in which at least one protein selected from the group consisting of PON1, MMRN1, MBL2, and CNDP1 is present in a higher or lower concentration compared to a non-cognitively impaired control sample. For example, the Alzheimer's disease patient may have an extracellular vesicle content in which at least one protein selected from the group consisting of PON1, MMRN1, MBL2, and CNDP1 is present in a higher concentration compared to a non-cognitively impaired control sample. In yet a further embodiment, the Alzheimer's disease patient may have an extracellular vesicle content in which at least one protein selected from the group consisting of CNDP1 and MMRN1 is present in a higher or lower concentration compared to a non-cognitively impaired control sample. For example, the Alzheimer's disease patient may have an extracellular vesicle content in which at least one protein selected from the group consisting of CNDP1 and MMRN1 is present in a higher concentration compared to a non-cognitively impaired control sample.

In certain embodiments, the Alzheimer's disease patient may have an extracellular vesicle content in which at least one protein selected from the group consisting of FERMT3, CAT, ALAD, SERPINF2, vWF, FCN2, and F13A1 is present in a higher or lower concentration compared to a non-cognitively impaired control sample. For example, the Alzheimer's disease patient may have an extracellular vesicle content in which at least one protein selected from the group consisting of FERMT3, CAT, ALAD, SERPINF2, vWF, FCN2, and F13A1 is present in a lower concentration compared to a non-cognitively impaired control sample. In further embodiments, the Alzheimer's disease patient may have an extracellular vesicle content in which at least one protein selected from the group consisting of FERMT3, CAT, SERPINF2, and FCN2 is present in a higher or lower concentration compared to a non-cognitively impaired control sample. In further embodiments, the Alzheimer's disease patient may have an extracellular vesicle content in which at least one protein selected from the group consisting of FERMT3, CAT, SERPINF2, and FCN2 is present in a lower concentration compared to a non-cognitively impaired control sample.

In certain embodiments, the Alzheimer's disease patient may have an extracellular vesicle content in which at least one protein selected from the group consisting of ECM1, VCL, RAP1B, KLKB1, and PARVB is present in a higher or lower concentration compared to a non-cognitively impaired control sample. For example, the Alzheimer's disease patient may have an extracellular vesicle content in which at least one protein selected from the group consisting of ECM1, VCL, RAP1B, KLKB1, and PARVB is present in a lower concentration compared to a non-cognitively impaired control sample. In further embodiments, the Alzheimer's disease patient may have an extracellular vesicle content in which at least one protein selected from the group consisting of RAP1B, VCL, and PARVB is present in a higher or lower concentration compared to a non-cognitively impaired control sample. In further embodiments, the Alzheimer's disease patient may have an extracellular vesicle content in which at least one protein selected from the group consisting of RAP1B, VCL, and PARVB is present in a lower concentration compared to a non-cognitively impaired control sample.

In some embodiments, the Alzheimer's disease patient is determined to have an extracellular vesicle content different from a non-cognitively impaired control sample. For example, the Alzheimer's disease patient is determined to have an extracellular vesicle content in which at least one of the proteins listed in Table 1 (of Example 2) is present in a higher or a lower concentration compared to a non-cognitively impaired control sample. In some embodiments, the at least one protein indicated as down regulated (in Table 1) is present in a lower concentration compared to a non-cognitively impaired control sample. In some embodiments, the at least one protein indicated as up regulated (in Table 1) is present in a higher concentration compared to a non-cognitively impaired control sample.

By “determined to have, the present specification means that the patient is subjected to some form of diagnostic testing prior to being administered the depleted blood fraction of the present invention, or prior to being treated by the method of the present invention.

In other embodiments, the Alzheimer's disease patient is determined to have an extracellular vesicle content in which at least one protein selected from the group consisting of CLU, HSPA5, HSP90B1, CALR, PLPT, and SOD2 is present in a higher or a lower concentration compared to a non-cognitively impaired control sample. For example, the Alzheimer's disease patient is determined to have an extracellular vesicle content in which at least one protein selected from the group consisting of CLU, HSPA5, HSP90B1, CALR, PLPT, and SOD2 is present in a higher concentration compared to a non-cognitively impaired control sample. In other embodiments, the Alzheimer's disease patient may have an extracellular vesicle content in which at least one protein selected from the group consisting of CLU and SOD2 is determined to be present in a higher or a lower concentration compared to a non-cognitively impaired control sample. For example, the Alzheimer's disease patient may have an extracellular vesicle content in which at least one protein selected from the group consisting of CLU and SOD2 is determined to be present in a higher concentration compared to a non-cognitively impaired control sample.

In other embodiments, the Alzheimer's disease patient is determined to have an extracellular vesicle content in which at least one protein selected from the group consisting of PON1, MMRN1, MBL2, and CNDP1 is present in a higher or lower concentration compared to a non-cognitively impaired control sample. For example, the Alzheimer's disease patient is determined to have an extracellular vesicle content in which at least one protein selected from the group consisting of PON1, MMRN1, MBL2, and CNDP1 is present in a higher concentration compared to a non-cognitively impaired control sample. In yet a further embodiment, the Alzheimer's disease patient is determined to have an extracellular vesicle content in which at least one protein selected from the group consisting of CNDP1 and MMRN1 is present in a higher or lower concentration compared to a non-cognitively impaired control sample. For example, the Alzheimer's disease patient is determined to have an extracellular vesicle content in which at least one protein selected from the group consisting of CNDP1 and MMRN1 is present in a higher concentration compared to a non-cognitively impaired control sample.

In certain embodiments, the Alzheimer's disease patient is determined to have an extracellular vesicle content in which at least one protein selected from the group consisting of FERMT3, CAT, ALAD, SERPINF2, vWF, FCN2, and F13A1 is present in a higher or lower concentration compared to a non-cognitively impaired control sample. For example, the Alzheimer's disease patient is determined to have an extracellular vesicle content in which at least one protein selected from the group consisting of FERMT3, CAT, ALAD, SERPINF2, vWF, FCN2, and F13A1 is present in a lower concentration compared to a non-cognitively impaired control sample. In further embodiments, the Alzheimer's disease patient is determined to have an extracellular vesicle content in which at least one protein selected from the group consisting of FERMT3, CAT, SERPINF2, and FCN2 is present in a higher or lower concentration compared to a non-cognitively impaired control sample. In further embodiments, the Alzheimer's disease patient is determined to have an extracellular vesicle content in which at least one protein selected from the group consisting of FERMT3, CAT, SERPINF2, and FCN2 is present in a lower concentration compared to a non-cognitively impaired control sample.

In certain embodiments, the Alzheimer's disease patient is determined to have an extracellular vesicle content in which at least one protein selected from the group consisting of ECM1, VCL, RAP1B, KLKB1, and PARVB is present in a higher or lower concentration compared to a non-cognitively impaired control sample. For example, the Alzheimer's disease patient is determined to have an extracellular vesicle content in which at least one protein selected from the group consisting of ECM1, VCL, RAP1B, KLKB1, and PARVB is present in a lower concentration compared to a non-cognitively impaired control sample. In further embodiments, the Alzheimer's disease patient is determined to have an extracellular vesicle content in which at least one protein selected from the group consisting of RAP1B, VCL, and PARVB is present in a higher or lower concentration compared to a non-cognitively impaired control sample. In further embodiments, the Alzheimer's disease patient is determined to have an extracellular vesicle content in which at least one protein selected from the group consisting of RAP1B, VCL, and PARVB is present in a lower concentration compared to a non-cognitively impaired control sample.

It should be appreciated by those skilled in the art that the specific embodiments disclosed within paragraphs [0020]-[0034] should not be read in isolation, and that the present specification intends for these embodiments to be disclosed in combination with other embodiments as opposed to being disclosed individually. For example, each of the embodiments disclosed in paragraphs [0009]-[0019] is to be read as being explicitly combined with each of the embodiments in paragraphs [0020]-[0034], or any permutation of 2 or more of the embodiments disclosed therein.

### Plasma Exchange Volumes

Typically, a patient's total blood volume is calculated as per Nadler's formula Nadler S B, Hidalgo J H. Bloch T. Prediction of blood volume in normal human adults. Surgery. 1962:51(2):224-32), reproduced below:

### Patient Total Blood Volume (ml):

$$Male = ( 0.0 \; 06012 \times H 3 ) / ( 14.6 \times W ) + 604$$

$$Female = ( 0. 0 \; 05835 \times H 3 ) / ( 15 \times W ) + 183$$

$$H = height in inches , W = weight in pounds .$$

Plasma exchange procedures report the quantity of blood/plasma processed in terms of a patient's total blood volume. Processing 1 Volume equates to processing the patient's total blood volume as determined by Nadler's formula. Similarly, processing 1.5 volumes equates to processing “1.5× the patient's total blood volume” as determined by Nadler's formula. Naturally, volumes over 1 result in some of the patient's blood volume being processed more than once.

Plasma exchanges can also be reported in terms of plasma volumes, eg 1 plasma volume. Given that plasma accounts for about 55% of total blood volume the two naming systems are interrelated and ultimately centre upon the total blood volume calculation.

The present invention envisages processing from about 0.25 to about 2 blood volumes by plasma exchange. In some embodiments, from about 0.25 to about 0.5 blood volumes may be subjected to plasma exchange. For example, from about 0.3 to about 0.4 blood volumes may be subjected to plasma exchange. In one embodiment, about 0.33 blood volumes may be subjected to plasma exchange.

In other embodiments, from about 0.75 to about 2 blood volumes may be subjected to plasma exchange. For example, from about 1 to about 2 blood volumes may be subjected to plasma exchange. Such as, from about 1 to about 1.5 blood volumes may be subjected to plasma exchange. In one embodiment, about 1 blood volume may be subjected to plasma exchange.

The skilled person will appreciate that aside from Nadler's formula other less utilised formulae and formulae centred around blood volume averages for a particular range of body weight can also be utilised to determine a patient's total blood volume. Such alternative methodologies are also within the scope of the present invention. For the purposes of the present invention, the relevant variable is blood volume regardless of the method utilised to calculate same. Minor variances in the quantum of blood volume arising from the use of different formulae will not have an effect on the efficacy of the present invention.

In one embodiment, the patient diagnosed with the condition may have from about 10% to about 95% of their plasma removed from their blood as part of the plasma exchange procedure. In some embodiments, from about 10% to about 50% of their plasma may be removed from their blood. In other embodiments, from about 10% to about 40% of their plasma may be removed from their blood. For example, from about 20% to about 40% of their plasma may be removed from their blood. In yet other embodiments, from about 15% to about 30% of their plasma may be removed from their blood.

In further embodiments, from about 50% to about 95% of their plasma may be removed from their blood. In other embodiments, from about 60% to about 95% of their plasma may be removed from their blood. For example, from about 60% to about 90% of their plasma may be removed from their blood. For example, from about 60% to about 85% of their plasma may be removed from their blood. In yet other embodiments, from about 60% to about 80% of their plasma may be removed from their blood. In certain embodiments, from about 60% to about 75% of their plasma may be removed from their blood.

In some embodiments, the patient is subjected to multiple iterances of plasma exchange. For example, each iterance of plasma exchange may occur within 1 to 45 days of the previous iterance. In other embodiments, each iterance of plasma exchange may occur within 1 to 30 days of the previous iterance. For example, each iterance of plasma exchange may occur within 1 to 15 days of the previous iterance. In some embodiments, each iterance of plasma exchange may occur within 1 to 7 days of the previous iterance. In certain embodiments, each iterance of plasma exchange may occur within 1 to 3 days of the previous iterance.

It should be appreciated by those skilled in the art that the specific embodiments disclosed within paragraphs [0036]-[0044] should not be read in isolation, and that the present specification intends for these embodiments to be disclosed in combination with other embodiments as opposed to being disclosed individually. For example, each of the embodiments disclosed in paragraphs [0036]-[0044] is to be read as being explicitly combined with each of the embodiments in paragraphs [0009]-[0035], or any permutation of 2 or more of the embodiments disclosed therein.

### Replacement Fluids

Depending on the blood volume processed by the plasma exchange procedure (and as a result the percentage of plasma removed from the patient's blood) a patient may receive replacement fluids following a plasma exchange procedure to avoid hypotension and peripheral oedema. Typically, larger blood volumes require the administration of a replacement fluid to compensate for the volumes of plasma removed from the patient's blood. Suitable non-limiting examples of replacement fluids include albumin preparations, and fresh frozen plasma diluted with saline. Smaller blood/plasma volumes, such as those equivalent to plasma donation volumes do not usually require replacement fluids.

Between 70 and 80% of the oncotic activity in normal human plasma is attributable to its albumin content, which lies in the range of around 35-50 g/L. Consequently, the volume of albumin to be administered as a replacement fluid can be readily calculated based on the blood volume subjected to plasma exchange/the volume of plasma removed from the patient's blood.

Accordingly, in certain embodiments the patient may be administered between 10 g and 60 g of albumin per Litre of plasma removed by the plasma exchange procedure. For example, the patient may be administered between 30 g and 50 g of albumin per Litre of plasma removed by the plasma exchange procedure.

An iso-oncotic solution of human serum albumin is the most common choice for plasma replacement in plasma exchange. Naturally, this replacement fluid strategy may lead to a transient decline in the levels of non-albumin plasma constituents including coagulation factors, immunoglobulins, transport proteins, and complement components. In practice, redistribution and re-synthesis keep most other plasma proteins in a satisfactory range. In some circumstances, augmentation therapy with other plasma constituents may be performed should deficiencies arise.

It should be appreciated by those skilled in the art that the specific embodiments disclosed within paragraphs [0046]-[0049] should not be read in isolation, and that the present specification intends for these embodiments to be disclosed in combination with other embodiments as opposed to being disclosed individually. For example, each of the embodiments disclosed in paragraphs [0046]-[0049] is to be read as being explicitly combined with each of the embodiments in paragraphs [0009]-[0045], or any permutation of 2 or more of the embodiments disclosed therein.

### BRIEF DESCRIPTION OF THE DRAWINGS

Additional features and advantages of the present invention will be made clearer in the appended drawings, in which:

FIG. 1 plots the results of size exclusion chromatography (SEC) and flow cytometry analysis and identifies the presence of extracellular vesicles (EVs) in plasma after standard centrifugation, total plasma exchange (TPE) and low volume plasma exchange (LVPE);

FIG. 2 is a graph illustrating extracellular vesicle concentrations and sizes in isolated fractions as measured by nanoparticle tracking analysis;

FIG. 3 plots the relative amounts of EV-associated proteins including TSG101, Flotillin and Syntenin from plasma obtained by standard centrifugation, LVPE and TPE as detected by Western Blot;

FIG. 4 depicts the structure and size distribution of extracellular vesicles in plasma obtained by standard centrifugation, LVPE and TPE using CRYO-TEM;

FIGS. 5A-5E depict the results of a series of analytical tests on EVs isolated from Alzheimer's Disease patients and healthy aged-matched controls;

FIG. 6 is a Principal Components Analysis (PCA) plot of the differentially regulated proteins identified in Table 1;

FIG. 7 shows the effect of CTRL and AD plasma-EVs in BV2 microglial cell activation. Upper panels (A) correspond to nitrite determination of the CTRL and AD plasma-EVs, and lower panels (B) show the negative and positive controls. Bars represent the mean SEM;

FIG. 8 shows nitrite concentration considering the different amounts of CTRL- and AD-EVs at different exposure times with BV2 microglial cells. Bars represent the mean±SEM;

FIG. 9 shows the effect of CTRL and AD plasma-EVs in BV2 microglial cell activation. A) Nitrite determination of the CTRL and AD plasma-EVs and B) show the negative and positive controls. Bars represent the mean±SEM. To determine statistical differences between groups non-parametric Kruskal-Wallis test with Dunn's multiple comparisons test was applied (significant when, *P<0.05); and

FIG. 10 shows nitrite concentration considering the different amounts of CTRl- and AD-EVs at different exposure times with BV2 microglial cells. A total of 25.000 BV2 cells were exposed to different quantities of EVs (5 μg, 10 μg and 20 μg of proteins) and then nitrite concentration was determined following 24 h of exposure. Bars represent the mean±SEM.

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1—Assessing the Impact of Plasma Exchange on Extracellular Vesicle (EV) Depletion from Plasma

A study was performed to analyse the impact of plasma exchange on extracellular vesicle isolation/removal. In order to carry out the study, plasma was obtained from five blood donors (n=5) using standard centrifugation, and two different apheresis/plasmapheresis devices.

Blood was obtained from five different human donors. From each of the five donors, two whole blood bags (approximately 250 ml each) were filled to ensure homogenous sampling. Plasma samples were obtained from the donors' blood using standard centrifugation [A], the Spectra Optia® Apheresis System (Terumo BCT, Inc.) [B] and the Autopheresis-C™ Plasmapheresis System (Fresenius Kabi) [C]; further particulars of which are outlined below. Whilst both apheresis systems separate plasma from blood, they do so utilising different mechanical principals.

### Plasma Obtained by Standard Centrifugation

For donor 1, 15 ml of blood from each of blood bag I and blood bag II were pooled into a 50 ml Falcon tubes. The resulting 30 ml pool of human blood were then subjected to centrifugation for 10 min at 500×g followed by 15 minutes at 2,500×g using the Centrifuge 5702R (Eppendorf, Hamburg, Germany). This process was repeated for each of the remaining donors 2 to 5. On average, 14 to 16 ml of plasma were isolated from the blood samples, aliquoted in cryotubes and stored at −80° C. until evaluation.

### Plasma Obtained Using the Spectra Optia® Apheresis System (Terumo BCT Inc.)

The Spectra Optia Apheresis System is an automatic and continuous blood component separator that uses centrifugation and optical detection to perform plasma separation procedures. It is generally used for large volume separations (>11) but can also be used with lower volume separations (<900 ml).

For donor 1, 200 ml of blood from blood bag I was processed using the Spectra Optia Apheresis System. This process was repeated for each of the remaining donors 2 to 5. On average, 70 to 110 ml of plasma were isolated from the blood samples, aliquoted in cryotubes and stored at −80° C. until evaluation.

### Plasma Obtained Using the Autopheresis-C™ Plasmapheresis System (Fresenius Kabi)

The Autopheresis-C system is a blood component separator that uses utilizes a spinning elongate membrane combined with a tangential filtration to mechanically separate plasma from blood. It is generally used for lower volume separations up to a maximum of 880 ml.

For donor 1, 200 ml of blood from blood bag II was processed using the Autopheresis-C system. This process was repeated for each of the remaining donors 2 to 5. On average, 50 to 80 ml of plasma were isolated from the blood samples, aliquoted in cryotubes and stored at −80° C. until evaluation.

### Isolation and Processing of EVs

To assess the impact of plasma exchange on extracellular vesicle isolation, extracellular vesicles from the obtained plasma samples were separated by size exclusion chromatography (SEC).

Two millilitres of sample (plasma) were used for isolating extracellular vesicles. Columns were manually loaded with 10 ml Sepharose™ CL-2B, and once tempered at room temperature, samples were carefully loaded onto the columns. Different fractions (approximately 35 fractions, 500 μL each) were progressively collected using PBS1X as elution buffer. All fractions were firstly characterized for their protein concentration using a Nanodrop® UV-Vis spectrophotometer (Abs 280 nm).

Low protein content fractions (namely, lacking albumin), as determined by Nanodrop® UV-Vis spectrophotometry, were analysed by flow cytometry to determine extracellular vesicle enriched fractions. EVs were detected by the presence of three classical extracellular vesicle markers: tetraspanins CD63, CD9 and CD81.

The four fractions showing the highest MFI fold change were pooled and identified as the extracellular vesicle-enriched fraction. Additionally, the four fractions with the highest protein concentration were pooled and identified as the protein-enriched fraction. Finally, the four fractions in-between the protein-enriched and extracellular vesicle-enriched fractions were pooled and identified as the intermediate-fraction. Then, total protein concentration from the different pooled fractions was determined by bicinchoninic acid assay (MicroBCA™ Protein Assay kit, ThermoFisher Scientific) following the manufacturer's instructions.

Flow cytometry with extracellular vesicle markers (vide supra) was used to identify extracellular vesicle enriched fractions, which were analysed for the presence of additional extracellular vesicle markers by western blot. Antibodies against TSG101 (ab30871, abeam), Flotilin-1 (ref: 610820, BD Biosciences) and Syntenin (ab133267, abeam) were used following a standard protocol.

From each of the three groups (standard centrifugation, Spectra Optia and Autopheresis-C) twenty micrograms from the extracellular vesicle-enriched pool was subjected to size distribution analysis and the estimated extracellular vesicle concentration was determined using Nanoparticle Tracking Analysis (NTA) (NanoSight NS300, Malvern Instruments Limited) following the manufacturer's instructions. Samples were diluted 1:40 with sterile and filtered PBS and, each sample was measured three times. The mean of the three replicates was considered as the final measurement. A volume of 3-5 μl of the different fractions (EV-enriched fraction, protein-enriched fraction and intermediate-fraction) were pooled for CRYO-TEM analysis. Size distribution quantification was done by calculating the diameter of all vesicles found in the images obtained by CRYO-TEM. At least, 50 vesicles were counted for each sample.

### Discussion of Results

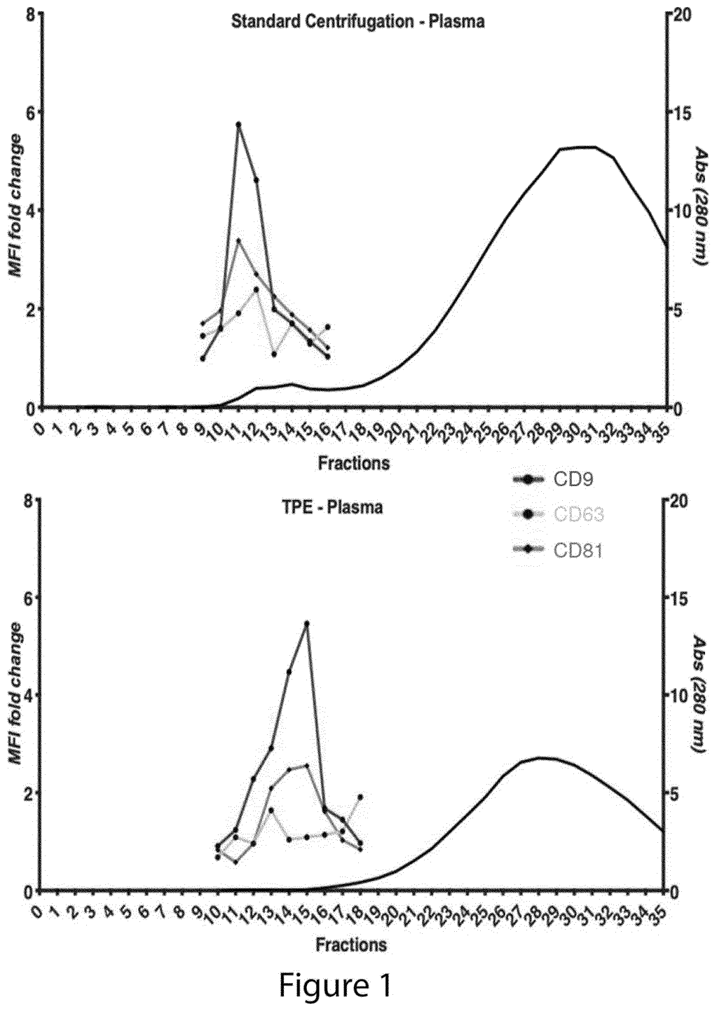

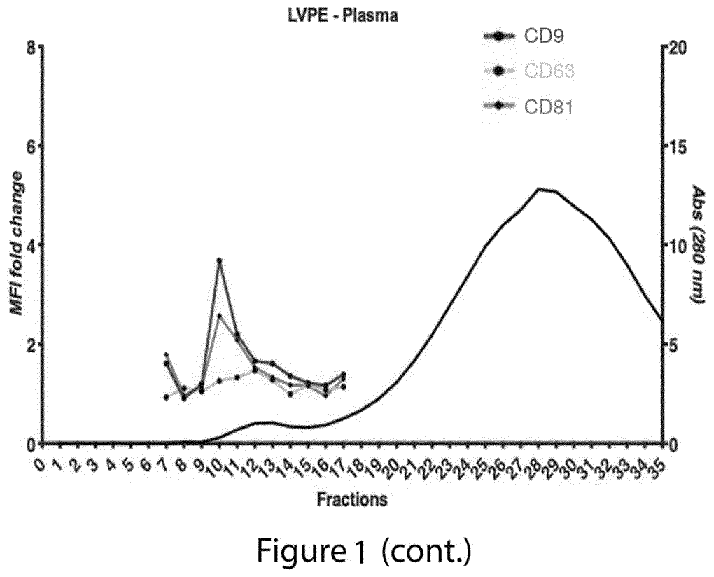

As shown in FIG. 1, SEC followed by flow cytometry facilitated the identification of extracellular vesicle enriched fractions in plasma obtained through each of the procedures disclosed in paras. [0067]-[0071]. Results from each of the plasmapheresis procedures are comparable to the fractions obtained from the standard centrifugation cohort.

FIG. 1 evidences the presence of tetraspanins CD81, CD9 and CD63 (which are representative extracellular vesicle biomarkers) in a similar sample range for each of the three groups (Centrifuge, Spectra Optia, and Autopheresis-C).

As disclosed in [0071] supra, nanoparticle tracking analysis was utilised to determine extracellular particle concentrations and size distribution in the extracellular vesicle enriched fractions from the donors' samples.

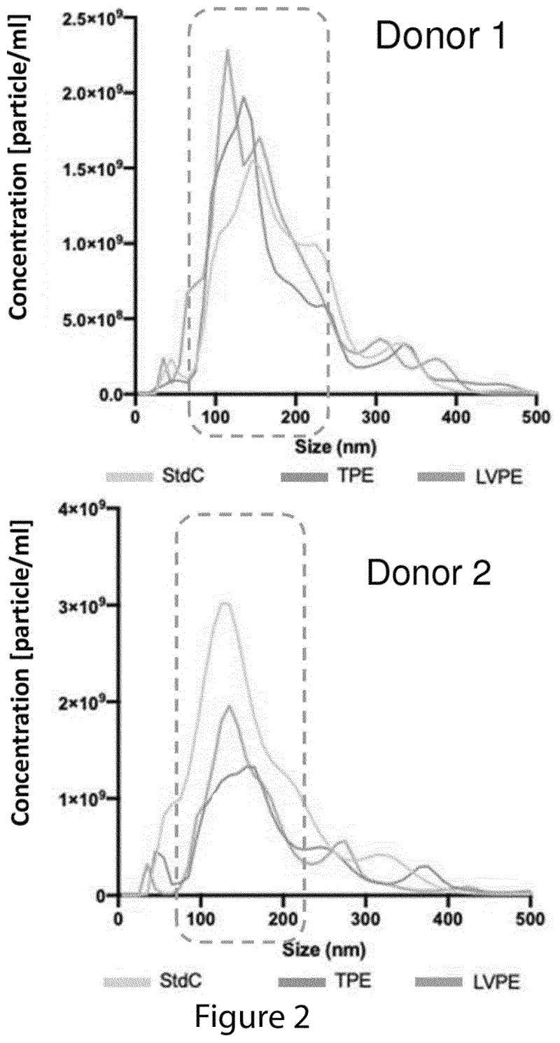

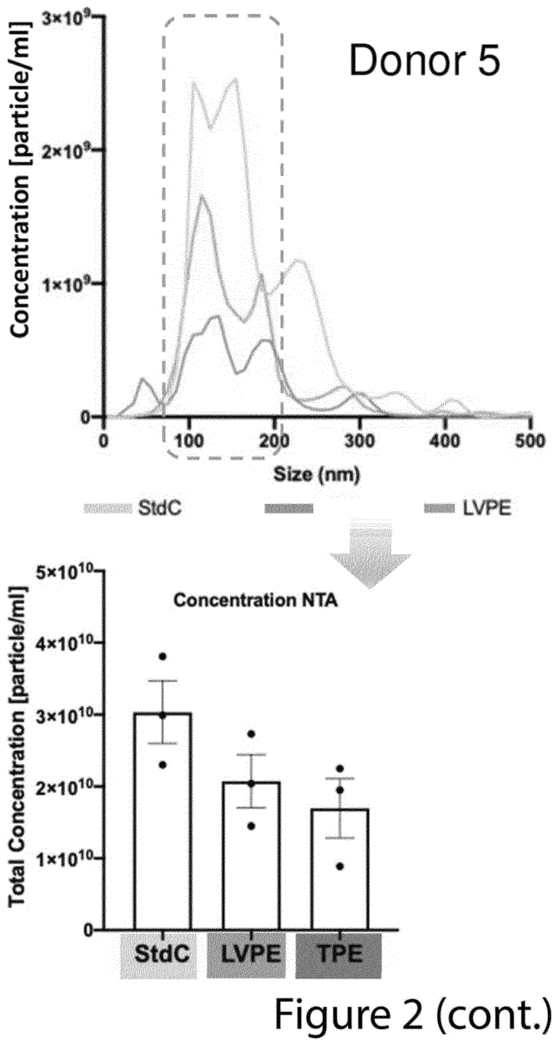

The plots in FIG. 2 reveal that extracellular vesicle enriched fractions have comparable particle concentration (particle/ml) and size distribution in plasma obtained by the different methodologies: standard centrifugation (“StdC”), Spectra Optia processing (“TPE”) and Autopheresis-C processing (“1 VPE”) in the Figures respectively. The concentrations found were typically in the range of 10⁹-10¹² particles/ml.

As disclosed in [0070] supra, the molecular composition of the extracellular vesicles was determined by Western Blot for further confirmation that the isolated species were in fact extracellular vesicles. Samples were compared to a positive control provided by a SH-SY5Y cell line lysate known to contain the protein markers.

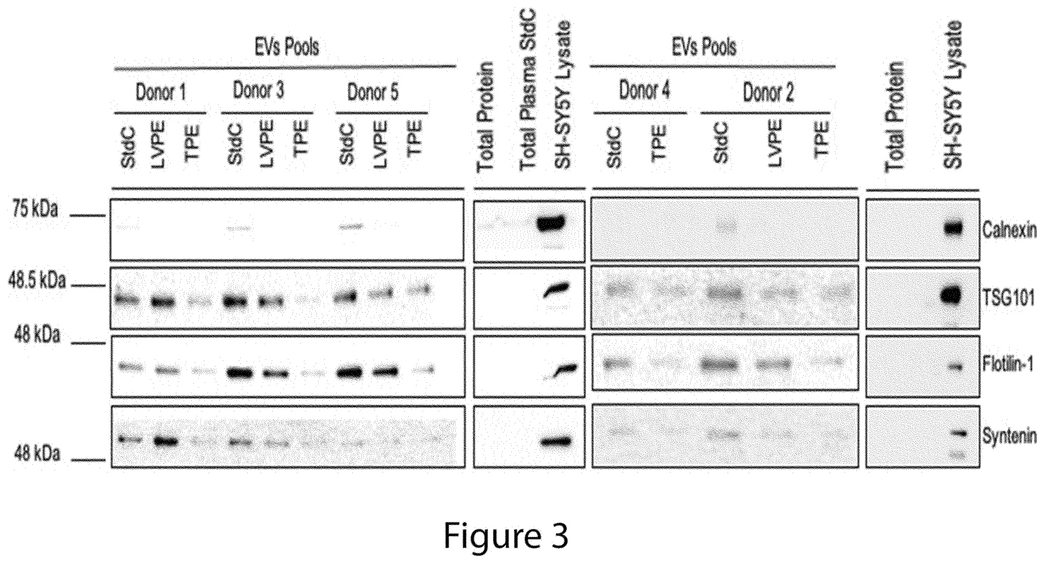

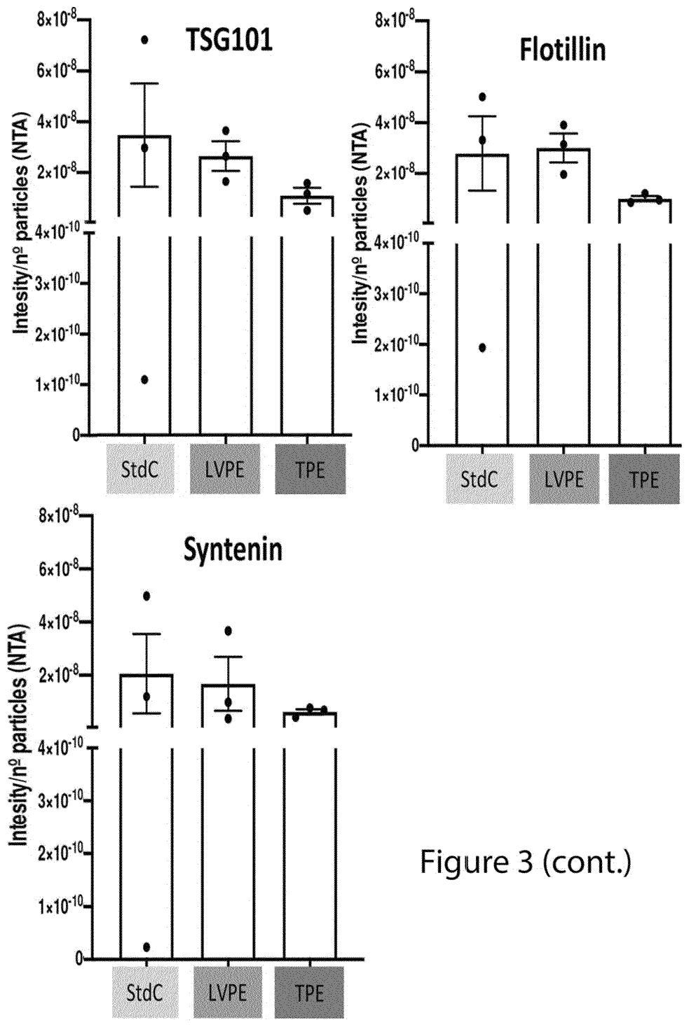

From FIG. 3 it is clearly demonstrated that the additional extracellular vesicle markers (TSG101, Flotilin-1, and Syntenin) were determined to be present by Western Blot in the extracellular vesicle enriched fractions from plasma obtained by standard centrifugation, LVPE and TPE. The relative intensity of each biomarker in each of the standard centrifugation, TPE and LVPE groups is plotted as a bar chart in FIG. 3. The amounts are consistent across all three of standard centrifugation, TPE, and LVPE groups.

The different SEC fractions (EV-enriched fraction, protein-enriched fraction and intermediate-fraction) were subsequently analysed by CRYO-TEM microscopy. Vitrified specimens were prepared by placing 3 μL of a sample on a Quantifoil 1.2/1.3 TEM grid, blotted to a thin film and plunged into liquidethane-N2(I) in the Leica EM CPC cryowork station in the Centro Nacional de Biotecnologia (Madrid, Spain) and at Universitat Autonoma de Barcelona (UAB, Barcelona, Spain). The grids were transferred to a 626 Gatan cryoholder and maintained at −179° C. The grids were analyzed with a Jeol JEM 2011 transmission electron microscope operating at an accelerating voltage of 200 kV. Images were recorded on a Gatan Ultrascan 2000 cooled charge-coupled device (CCD) camera with the Digital Micrograph software package (Gatan). Size distribution quantification was done calculating the diameter of all vesicles found in the images. At least 50 vesicles were counted for each sample.

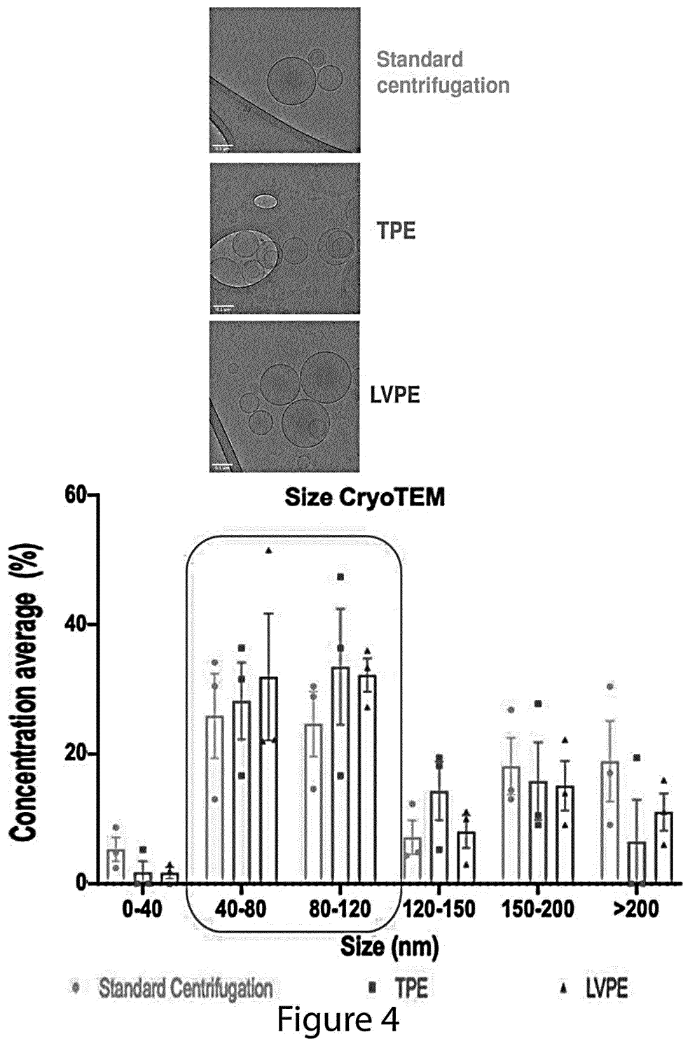

FIG. 4 illustrates the results from the CRYO-TEM analysis and demonstrates little variance in structure and size distribution of extracellular vesicles in plasma obtained by either standard centrifugation, LVPE or TPE. The majority of the extracellular vesicles were found to be in a size range of between 40 to 120 nm.

### Example 2—Identification of Proteins in EVs from Patients Suffering with Alzheimer's Disease Versus Healthy Controls

A proteomic analysis of EVs from the plasma samples from two different groups was performed: Alzheimer's Disease patients (n=10) and cognitively unimpaired age matched individuals (Controls, n=10). Alzheimer's samples were obtained from patients enrolled in a Phase II clinical study performed by Grifols (NCT00742417).

EVs were isolated from 1 ml of plasma according to an analogous procedure to that outlined in para. [0067]. Fractions were subsequently pooled into EV-enriched fractions, protein-enriched fractions, and intermediate fractions lying between the previous two categories. This process was completed for each of the n=10 Alzheimer's patients and n=10 age matched controls.

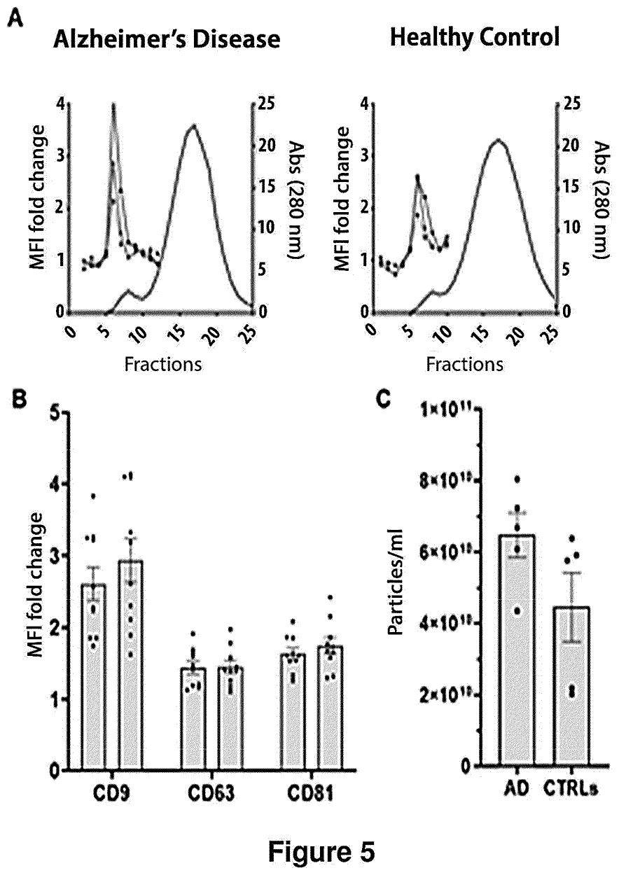

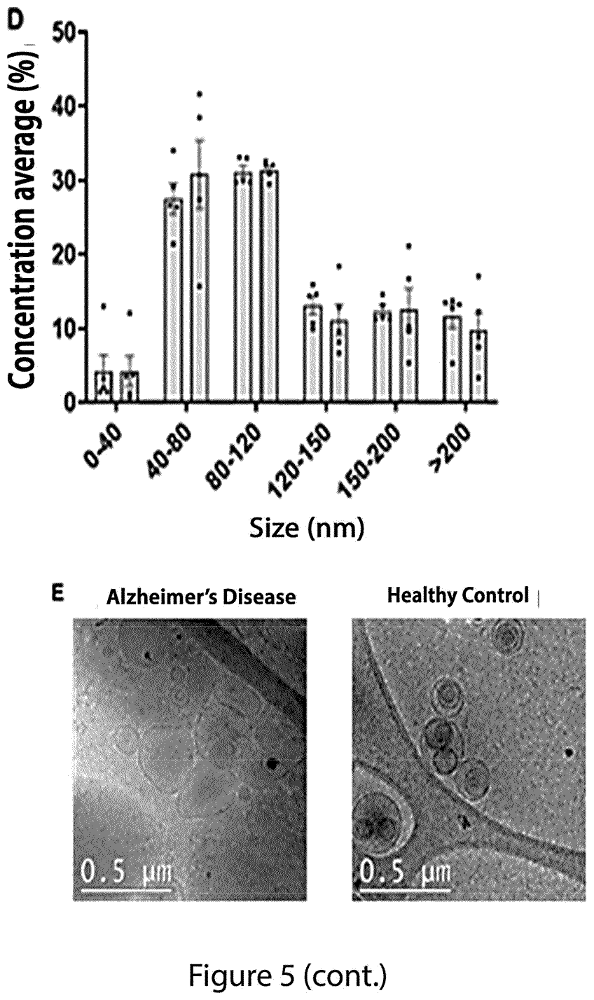

From FIGS. 5A-5E it is evident that the molecular structure of the EVs isolated from the plasma of Alzheimer's Disease (AD) patients versus age matched controls (CTRL) showed no significant differences between the two sample groups.

FIG. 5a provides a representative SEC profile of an AD and a CTRL sample, showing the fractions enriched in EV markers (MFI fold change on the Y-axis) and protein Nanodrop determinations at a wavelength of 280 nm (right hand side, Y-axis). FIG. 5B plots the MFI values of the EV-markers CD9, CD63 and CD81 in the EV-enriched pool of AD (left hand side) and CTRL (right hand side) cohorts (n=10). FIG. 5C provides a plot of particle concentration in EV-enriched pools measured by NTA. The size distribution of EVs using Cryo-TEM images from EV-enriched fraction from AD (n=5) and CTRLs (n=5) is provided in FIG. 5D. At least 150 EVs were measured in each sample. FIG. 5E illustrates representative Cryo-TEM images from an AD and a CTRL sample. Data in FIGS. 5B-5D are represented as the mean±SEM, and Mann Whitney test was applied to detect differences in the number of proteins identified in each plasma volume.

### Proteomics Protocol

0.5 ml of the pooled EV-enriched fraction was processed for proteomic analysis as outlined below (n=8 AD, n=10 CTRL). The proteomics workflow is based on in-gel proteolytic digestion of the sample, followed by liquid chromatography and mass spectrometry (LC-MS/MS), identification of peptides and proteins, and a corresponding downstream analysis.

Peptide and protein identification were performed with Proteome Discoverer™ (Thermo Fisher Scientific™), which determines abundance values at the peptide spectrum level. The abundances of the peptides are then summed to give the abundances at the protein level. The identification of a specific protein is also characterized by the coverage factor. Coverage designates the percentage of the protein that is coveraged by the identified peptides: the more peptides identified belonging to a specific protein, the higher is the associated coverage for this protein.

Once a protein is identified, it is quantified using MaxQuant software (Cox, J and Mann, M., 2008. MaxQuant enables high peptide identification rates, individualized p.p.b.-range mass accuracies and proteome-wide protein quantification. Nat Biotechnol, vol. 26:1367-72). MaxQuant integrates a set of algorithms related to peak detection, scoring peptides, mass calibration, m/z retention time plane, and database searches. For peptide and protein identification, fragments are searched in an organism specific sequence database (human, in this case), and they are scored by a probability-based approach termed a peptide score. For limiting the number of peak/fragment matches by chance a target-decoy-based false discovery rate (FDR) approach is utilized. The FDR is determined using statistical methods that account for multiple hypotheses testing.

Peptides identified with a high level of confidence (FDR=1%; peptide identifications being 99% accurate) are further quantified. An associated protein score, directly proportional to the individual peptide scores, measures the reliability of the identification. The higher this score, the more reliable the protein identification process.

Differential expression and statistical analyses were performed afterwards using Perseus Software (Tvanova, S. et al., 2016. The MaxQuant computational platform for mass spectrometry-based shotgun proteomics. Nature Protocols vol. 11: 2301). Proteins quantified with at least two unique peptides, a p-value lower than 0.05 and an absolute fold change of <0.77 (downregulation) or >1.3 (upregulation) on a linear scale were considered to be significantly differentially expressed between the two sample groups.

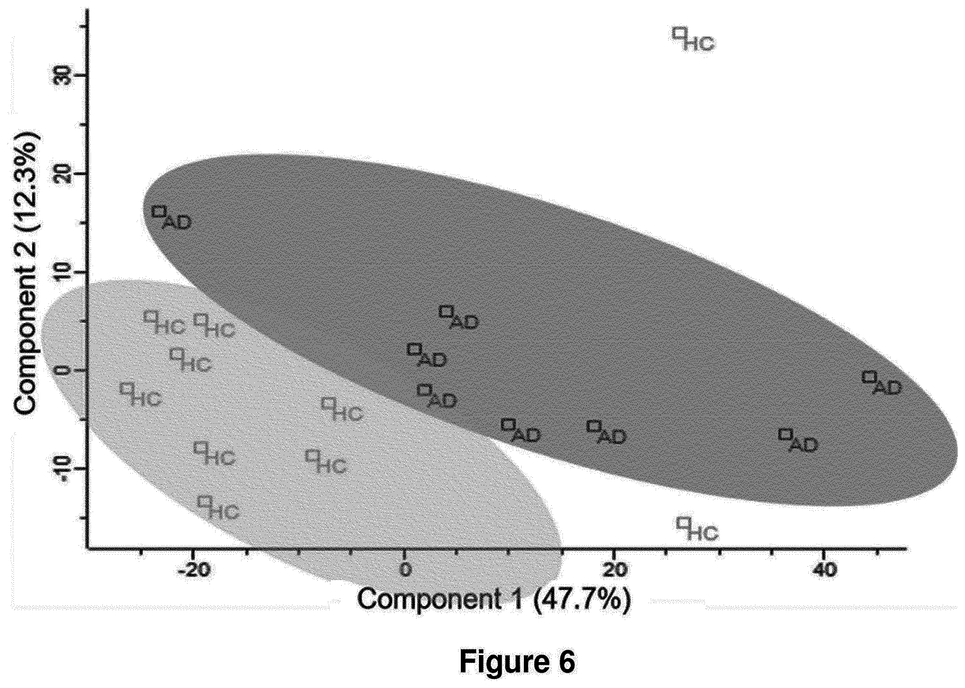

31 proteins (P<0.05) were found to be differentially contained in AD EVs versus healthy CTRL EVs. 16 Proteins were found to be under-represented and 15 proteins over-represented. The complete list of proteins can be found in Table 1. A Principal Component Analysis (PCA) using the proteins listed in Table 1 shows a defined separation between the two sample groups (FIG. 6).

The specifically dysregulated proteins were analyzed using EnrichR online tool (Xie, Z., et al. 2021. Gene Set Knowledge Discovery with Enrichr. Curr Protoc. vol. 1(3):e90) and GTex Portal (Genotype-Tissue Expression project, https://gtexportal.org/home/) for pathway related analyses.

EnrichR uniquely integrates knowledge from many high-profile projects to provide synthesized information about mammalian genes and proteins sets. The analysis of downregulated proteins in AD revealed a significant overrepresentation in proteins related to bloodstream and platelet biology according to Reactome 2016 database (Jassal, B. et al., 2020. The reactome pathway knowledgebase. Nucleic Acids Res, vol. 48(01):0498-0503). On the other hand, proteins over-represented in EVs from AD patients revealed biological pathways significantly related to protein folding, chaperones, and endoplasmic reticulum (ER) biology. ER is the main cell compartment involved in protein folding and secretion and is drastically affected in AD neurons, suggesting that circulating EVs can sense these perturbations.

To enhance the biological significance of the obtained results, differential expressed proteins in Table 1 were submitted to GTEx Portal and a multigene query for Tissue Gene Expression Profiles was performed.

Six out of the 15 overexpressed proteins in AD, namely CLU, HSPA5, HSP90B1, CALR, PLPT, and SOD2 were found as significantly expressed in brain tissues. Using String database v.11.5 (Szklarczyk, D. and Gable, A. L. et al., 2021. The STRING database in 2021: customizable protein-protein networks, and functional characterization of user-uploaded gene/measurement sets. Nucleic Acids Res, vol. 49(01):0605-612), these 6 proteins were functionally related and clustered as chaperones (FDR=4.85×10⁻⁵) and part of ER chaperone complex (FDR=6.85×10⁻⁶), and as involved in protein folding (FDR=2.64×10⁻⁵). These results correlate well with the output predicted using EnrichR.

Specifically, clusterin (CLU) prevents the aggregation of non-native proteins, including the formation of amyloid fibrils by amyloid precursor protein. Superoxide dismutase 2 (SOD2) is an essential enzyme for correct mitochondrial defence against superoxide. It converts toxic oxide species to less reactive molecules, such as hydrogen peroxide (H₂O₂). In neurodegenerative diseases, there is an overproduction of reactive oxygen species and an increase of cellular oxidative stress, where this enzyme may play an important role. Furthermore, SOD2 is increased in AD, co-localizing to senile plaques and dystrophic neurites indicating a firm association between free-radical mediated injury and the disease neuropathology.

Of the proteins indicated as downregulated in the EVs of AD patients (Table 1), a number of them have been reported as being potentially implicated in the pathology of AD. For example, each of FERMT3, CAT, ALAD, SERPINF2, vWF, FCN2, and F13A1 have been reported as having altered expression levels in AD patients.

TABLE 1

| Accession | Gene | Protein name | Regulation | FC1′ | P-value |
|---|---|---|---|---|---|
| Q86UX7-2 | FERMT3 | Fermitin family homolog 3 | Down | 0.086 | 0.0002 |
| A0A0B4J1Y8 | IGLV9-49 | Immunoglobulin lambda | Down | 0.110 | 0.001 |
| | | variable 9-49 | | | |
| Q13093 | PLA2G7 | Platelet-activating factor | Down | 0.150 | 0.0001 |
| | | acetylhydrolase | | | |
| Q16610 | ECM1 | Extracellular matrix protein 1 | Down | 0.155 | 0.023 |
| P01766 | IGHV3-13 | Immunoglobulin heavy | Down | 0.169 | 0.004 |
| | | variable 3-13 | | | |
| P04040 | CAT | Catalase | Down | 0.188 | 0.001 |
| P00748 | F12 | Coagulation factor XII | Down | 0.204 | 0.007 |
| P18206 | VCL | Vinculin | Down | 0.210 | 0.045 |
| P13716 | ALAD | Delta-aminolevulinic acid | Down | 0.291 | 0.049 |
| | | dehydratase | | | |
| P08697 | SERPINF2 | Alpha-2-antiplasmin | Down | 0.295 | 0.022 |
| P03952 | KLKB1 | Plasma kallikrein | Down | 0.318 | 0.001 |
| P61224-2 | RAP1B | Ras-related protein Rap-1b | Down | 0.338 | 0.023 |
| P04275 | VWF | von Willebrand factor | Down | 0.371 | 0.042 |
| Q15485 | FCN2 | Ficolin-2 | Down | 0.374 | 0.031 |
| Q9HBI1-3 | PARVB | Beta-parvin | Down | 0.413 | 0.042 |
| P00488 | F13A1 | Coagulation factor XIII A chain | Down | 0.441 | 0.022 |
| P01024 | C3 | Complement C3 | Up | 1.543 | 0.047 |
| P06310 | IGKV2-30 | Immunoglobulin kappa | Up | 1.749 | 0.038 |
| | | variable 2-30 | | | |
| P10909-6 | CLU | Clusterin | Up | 1.757 | 0.049 |
| P12259 | F5 | Coagulation factor V | Up | 1.768 | 0.037 |
| P27169 | PON1 | Serum | Up | 2.021 | 0.006 |
| | | paraoxonase/arylesterase 1 | | | |
| P55058-3 | PLTP | Phospholipid transfer protein | Up | 2.058 | 0.001 |
| P01834 | IGKC | Immunoglobulin kappa | Up | 2.197 | 0.023 |
| | | constant | | | |
| P07359 | GP1BA | Platelet glycoprotein Ib alpha | Up | 2.199 | 0.028 |
| | | chain | | | |
| Q96KN2 | CNDP1 | Beta-Ala-His dipeptidase | Up | 3.669 | 0.003 |
| P11021 | HSPA5 | Endoplasmic reticulum | Up | 3.823 | 0.002 |
| | | chaperone BiP | | | |
| Q13201 | MMRN1 | Multimerin-1 | Up | 3.947 | 0.019 |
| P14625 | HSP90B1 | Endoplasmin | Up | 4.821 | 0.004 |
| P11226 | MBL2 | Mannose-binding protein C | Up | 6.255 | 0.021 |
| P27797 | CALR | Calreticulin | Up | 9.246 | 0.0004 |
| P04179-4 | SOD2 | Superoxide dismutase | Up | 14.148 | 0.001 |

*FC, fold change

### Example 3—Functional Analysis of EVs. Effect of Control and AD Plasma-EVs in Microglia Activation

EVs were isolated from plasma samples obtained from control (CTRls) and Alzheimer's disease (AD) patients by size exclusion chromatography (SEC) as explained above.

In order to determine the effects of EVs, cultured naïve BV2 cells were treated with different amounts of EVs. For this approach, 6 ml pools obtained from four AD patients or four CTRls were used. EVs were isolated in three different SEC columns from AD and in different three columns for CTRls, and the resulting EVs-fractions were pooled obtaining a total of 6 ml of EV-enriched fraction from each type of sample.

Naïve BV2 cells were exposed to similar amounts of CTRl and AD-EVs, according to protein determination in each EVs pool. To evaluate the effects of the EV-enriched fraction from the two different cohorts, a nitrite determination after 90 min, 6 h, and 24 h of EV-treatment was done.

Flow cytometry results from plasma EV isolated by SEC showed the presence of EV marker CD9 at similar MFI levels in CTRl and AD samples. Protein concentration in the EV-enriched fraction was quantified by microBCA, with analogous determinations in AD and CTRl samples. For this quantification, % CV from Alzheimer's Disease and Healthy Control EV-fraction were: 33.07% and 34.36%, respectively.

In order to determine the biological effect of plasma-EVs from AD patients compared with CTRLs, different amounts of EVs (5 μg, 10 μg and 15 μg of protein) were incubated with naïve BV2 cells for 24 h and subsequently, nitrites were determined after 90 min, 6 hours, and 24 hours.

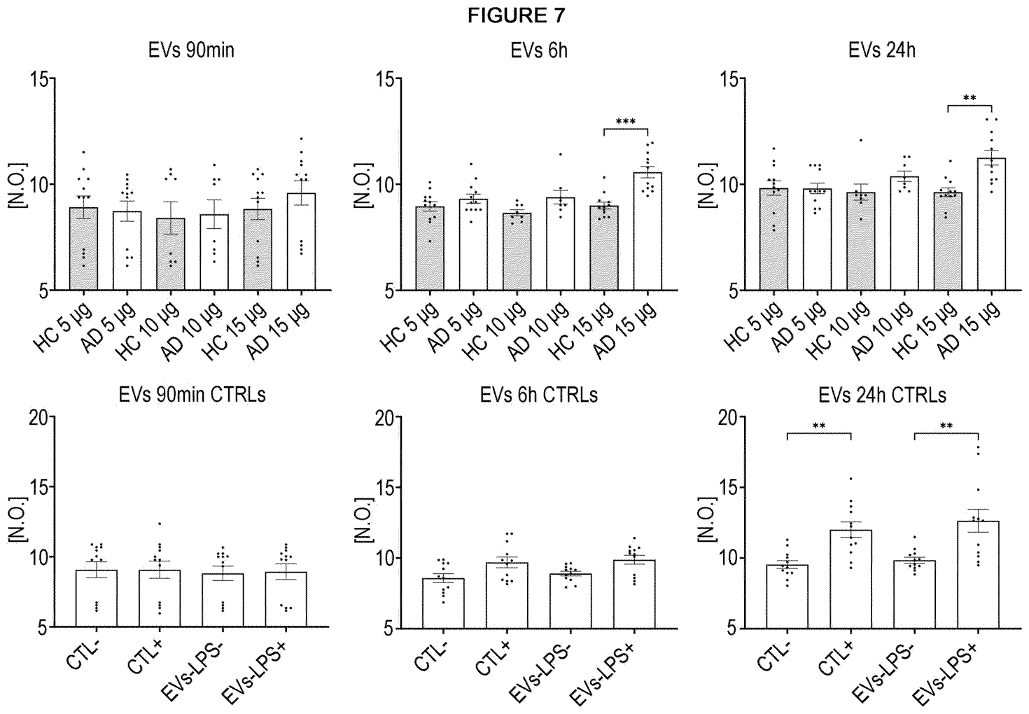

A significant activation of BV2 cells was detected following 24 h of exposure to 15 μg AD-plasma-EVs compared with CTRL-plasma-EVs (FIG. 7A). Activation was also detected following direct exposure to LPS and (LPS+BV2)-EVs, in agreement with the previous setting up (FIG. 7B). To determine statistical differences between groups non-parametric Kruskal-Wallis test with Dunn's multiple comparisons test was applied (significant when, **P<0.01; ***P<0.001).

A total of 50,000 BV2 cells were exposed to different quantities of EVs (5 μg, 10 μg and 15 μg of proteins) and then nitrite concentration was determined following 90 min, 6 h and 24 h of exposure.

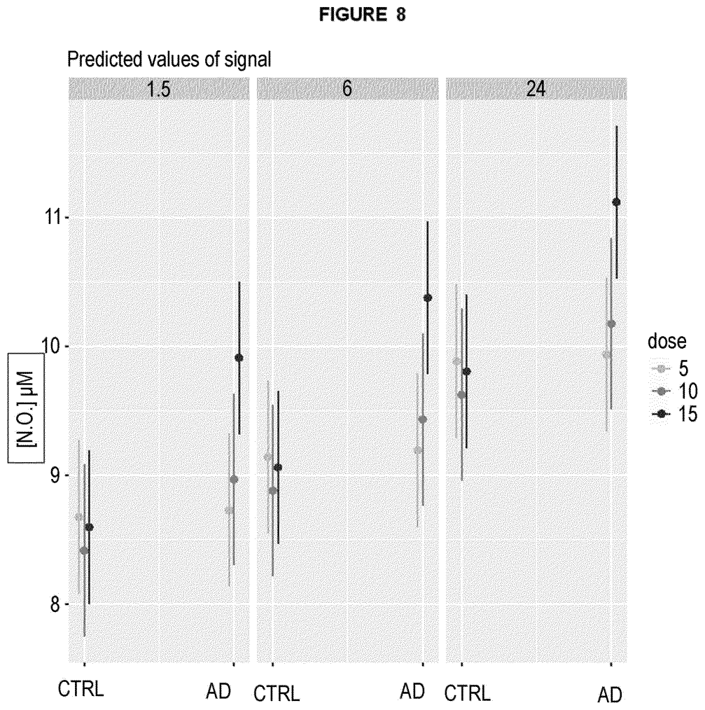

As it is shown in FIG. 8, applying a linear mixed effects model, a significant time effect independent of the EVs-doses or disease condition (AD or CTRL) was observed, meaning a progressive accumulation of nitrites over time in both conditions, as expected (FIG. 8, p-value<0.000001). In line with the previous analysis (FIG. 7), a significant dose-response in BV2 activation was only detected for AD plasma-EVs (FIG. 8, p-value=0.0068).

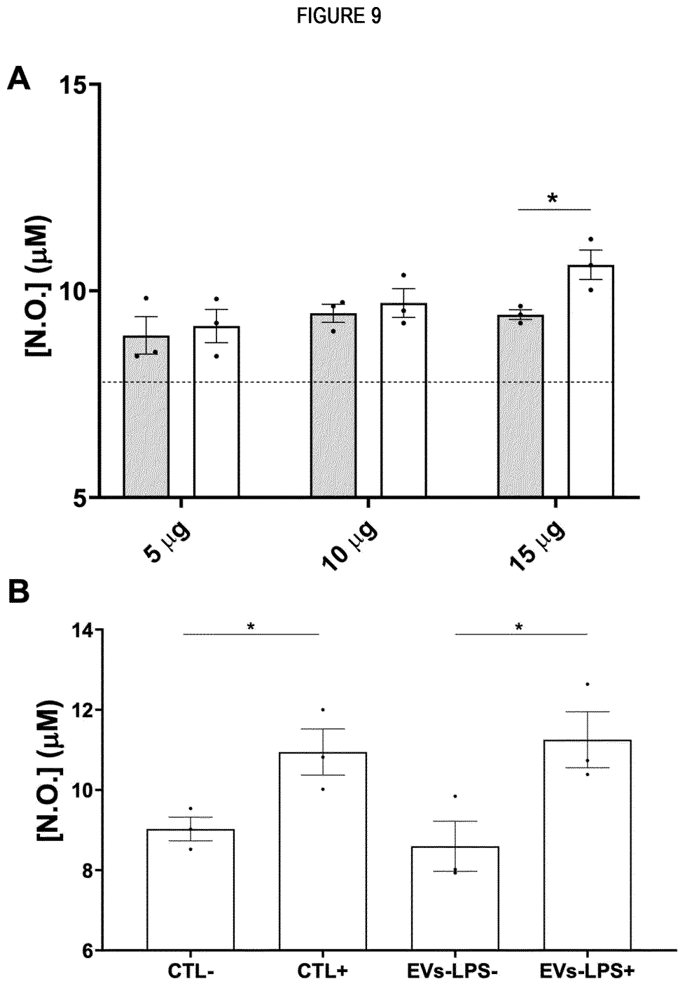

To validate the proinflammatory effect of AD plasma-EVs, independent CTRL and AD plasma samples and repeated NO2- measurement were used. Three different quantities of EVs (5 μg, 10 μg and 15 μg of protein) were exposed to 25,000 BV2 cells and nitrite was determined after 24 h. As it is shown in FIG. 9, a significant activation of BV2 cells was detected following 24 h of exposure to 15 μg AD-plasma-EVs compared with CTRL-plasma-EVs. Activation was also detected following direct exposure to LPS and (LPS+BV2)-EVs, in agreement with the previous setting up.

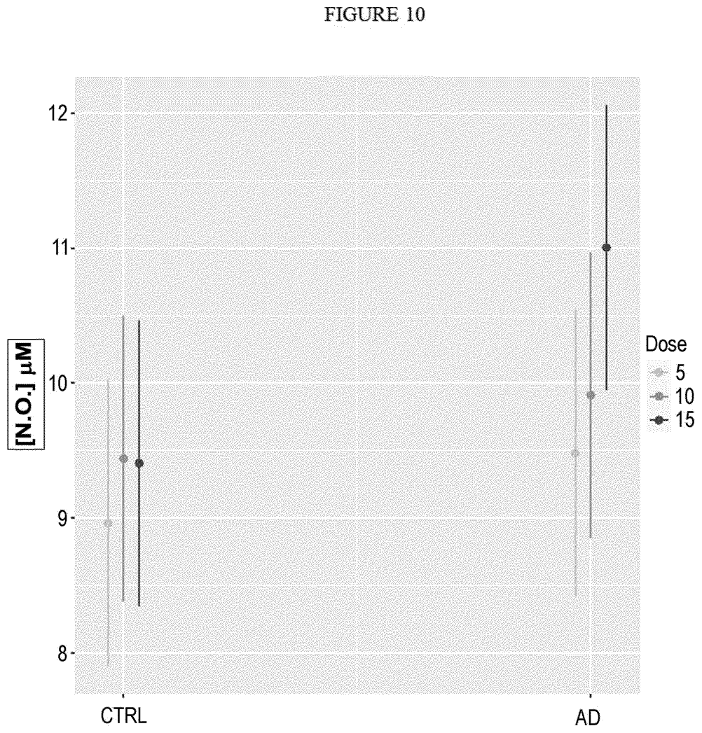

As it is shown in FIG. 10, applying a linear mixed effects model, a significant dose-response in BV2 activation was observed. It was only detected for AD plasma-EVs (p-value=0.0143).

The results show a significant activation (NO2- production) of BV2 cells following 24 h of exposure to AD-plasma-EVs compared with CTRL-plasma-EVs. Moreover, a significant dose-response effect in BV2 activation was only observed for AD plasma-EVs. These results indicate a proinflammatory cargo for AD EVs.

Overall, these results demonstrate that depletion of AD-plasma-EVs would reduce activation of microglia and consequently, would reduce inflammation. Considering that proliferation and activation of microglia in the brain is a prominent feature of neurodegenerative diseases, in particular AD, the extracellular vesicle depleted human blood fractions of the present invention are useful in the treatment of a neurodegenerative disease, in particular, AD.

1. A method for the treatment of a patient suffering from a condition selected from the group consisting of:
neurodegenerative disease, autoimmune disease, cardiovascular disease, renal disease, liver disease, and combinations thereof, the method comprising the step of subjecting the patient diagnosed with the condition to plasma exchange, so as the extracellular vesicle content from blood is depleted prior to being re-administered to said patient, and wherein this step of plasma exchange is performed in the absence of extracellular vesicle specific binding agent(s).

2. The method of claim 1, wherein from about 0.25 to about 2 blood volumes of the patient diagnosed with the condition is subjected to plasma exchange.

3. The method of claim 1, wherein from about 0.3 to about 0.4 blood volumes of the patient diagnosed with the condition is subjected to plasma exchange.

4. The method of claim 1, wherein from about 1 to about 1.5 blood volumes of the patient diagnosed with the condition is subjected to plasma exchange.

5. The method of claim 1, wherein from about 10% to about 95% of the plasma is removed from the blood of the patient diagnosed with the condition during the plasma exchange procedure.

6. The method of claim 5, wherein from about 10% to about 40% of the plasma is removed from the blood of the patient diagnosed with the condition during the plasma exchange procedure.

7. The method of claim 5, wherein from about 60% to about 95% of the plasma is removed from the blood of the patient diagnosed with the condition during the plasma exchange procedure.

8. The method of claim 1, wherein the patient is subjected to multiple iterances of plasma exchange, wherein each iterance of plasma exchange occurs within 1 to 45 days of the previous iterance of plasma exchange.

9. The method of claim 8, wherein each iterance of plasma exchange occurs within 1 to 7 days of the previous iterance of plasma exchange.

10. The method of claim 1, wherein the patient suffers from a neurodegenerative disease.

11. The method of claim 10, wherein the neurodegenerative disease is selected from the group consisting of Parkinson's disease, synucleinopathies, Alzheimer's disease, Mild Cognitive Impairment, Diffuse Lewy body disease, Dementia with Lewy bodies type, amyotrophic lateral sclerosis, Pick's disease, tauopathies, trinucleotide repeat expansion diseases such as Huntington's disease and spinocerebellar ataxias, Creutzfeldt-Jakob disease, frontotemporal dementia, and combinations thereof.

12. The method of claim 10, wherein the neurodegenerative disease is selected from the group consisting of Parkinson's disease, Alzheimer's disease, and Huntington's disease.

13. The method of claim 10, wherein the neurodegenerative disease is Alzheimer's disease.

14. The method of claim 1, wherein the patient suffers from Alzheimer's disease and has an extracellular vesicle content different from a non-cognitively impaired control sample.

15. The method of claim 14, wherein the patient suffers from Alzheimer's disease and has an extracellular vesicle content including at least one of the proteins listed in Table 1 at a higher or lower concentration when compared to a non-cognitively impaired control sample.

16. The method of claim 14, wherein the patient suffers from Alzheimer's disease and has an extracellular vesicle content including at least one protein selected from the group consisting of CLU, HSPA5, HSP90B1, CALR, PLPT, and SOD2 at a higher concentration when compared to a non-cognitively impaired control sample.

17. The method of claim 14, wherein the patient suffers from Alzheimer's disease and has an extracellular vesicle content including at least one protein selected from the group consisting of CLU and SOD2 at a higher concentration when compared to a non-cognitively impaired control sample.

18. The method of claim 14, wherein the patient suffers from Alzheimer's disease and has an extracellular vesicle content including at least one protein selected from the group consisting of PON1, MMRN1, MBL2, and CNDP1 at a higher concentration when compared to a non-cognitively impaired control sample.

19. The method of claim 14, wherein the patient suffers from Alzheimer's disease and has an extracellular vesicle content including at least one protein selected from the group consisting of FERMT3, CAT, ALAD, SERPINF2, vWF, FCN2, and F13A1 at a lower concentration when compared to a non-cognitively impaired control.

20. The method of claim 14, wherein the patient suffers from Alzheimer's disease and has an extracellular vesicle content including at least one protein selected from the group consisting of ECM1, VCL, RAP1 B, KLKB1, and PARVB at a lower concentration when compared to a non-cognitively impaired control sample.

21.-40. (canceled)
